SEQUENCE LISTING (4 sequences)

SEQ ID NO: 1 - DNA, 21 bp, synthetic construct
  source 1..21: mol_type = other DNA
cgcggatgagatccagaatac

SEQ ID NO: 2 - DNA, 20 bp, synthetic construct
  source 1..20: mol_type = other DNA
tcctcctcttcgtccttctt

SEQ ID NO: 3 - DNA, 26 bp, synthetic construct
  source 1..26: mol_type = other DNA
cagttccagcagatgtggaagcgttt

SEQ ID NO: 4 - RNA, 1110 nt, Homo sapiens
  source 1..1110: mol_type = mRNA
atgacgacctcaggcgcgctctttccaagcctggtgccaggctctcggggcgcctccaacaagtacttggtggagtttcgggcgggaaagatgtccctgaaggggaccaccgtgactccggataagcggaaagggctggtgtacattcagcagacggacgactcgcttattcacttctgctggaaggacaggacgtccgggaacgtggaagacgacttgatcatcttccctgacgactgtgagttcaagcgggtgccgcagtgccccagcgggagggtctacgtgctgaagttcaaggcagggtccaagcggcttttcttctggatgcaggaacccaagacagaccaggatgaggagcattgccggaaagtcaacgagtatctgaacaaccccccgatgcctggggcgctgggggccagcggaagcagcggccacgaactctctgcgctaggcggtgagggtggcctgcagagcctgctgggaaacatgagccacagccagctcatgcagctcatcggaccagccggccttggaggactgggtgggctgggggccctgactggacctggcctggccagcttactggggagcagtgggcctccagggagcagctcctcctccagctcccggagccagtcggcagcggtcaccccgtcatccaccacctcttccacccgtgccaccccagccccttctgctccagcagctgcctcagcaactagcccgagccccgcgcccagttccgggaatggagccagcacagcagccagcccgacccagcccatccagctgagcgacctccagagcatcctggccacgatgaacgtaccagccgggccagcaggcggccagcaagtggacctggccagtgtgctgacgccggagataatggctcccatcctcgccaacgcggatgtccaggagcgcctgcttccctacttgccatctggggagtcgctgccgcagaccgcggatgagatccagaataccctgacctcgccccagttccagcagatgtggaagcgtttgccaaagccatgcagaacaacgccaagcccgagcagaaagagggcgacacgaaggacaagaaggacgaagaggaggacatga

(19) United States
(12) Patent Application Publication
(10) Pub. No.: US 2025/0137057 A1
(43) Pub. Date: May 1, 2025

(54) ADRM1/RPN13 SPLICED VARIANT IN THE PROGNOSIS AND TREATMENT OF HEPATOCELLULAR CARCINOMA

(71) Applicant: The Chinese University of Hong Kong, Hong Kong (CN)

(72) Inventors: Nathalie WONG, Hong Kong (CN); Mingjing XU, Hong Kong (CN)

(21) Appl. No.: 18/495,414

(22) Filed: Oct. 26, 2023

(51) Int. Cl. C12Q 1/6886; A61K 31/454; A61K 31/4985; A61K 31/517; A61K 31/55; A61P 35/00

(52) U.S. Cl. CPC C12Q 1/6886; A61K 31/454; A61K 31/4985; A61K 31/517; A61K 31/55; A61P 35/00; C12Q 2600/106; C12Q 2600/156; C12Q 2600/158

(57) ABSTRACT

The subject invention pertains to a method of diagnosing and treating liver disease in humans. More specifically, the subject invention provides uses for ADRM1/Rpn13 spliced variant ΔEx9 (ADRM1-ΔEx9) as a biomarker for prediction of prognosis in HCC patients. By assessing the expression of the ADRM1-ΔEx9 isoform in patient biopsy or resected tumor, prognostic outcome can also be predicted. The invention also provides the use of ADRM1-ΔEx9 as a molecular marker to predict the response of HCC cells to the clinically approved PARP1 inhibitor Olaparib.

### SEQUENCE LISTING

The Sequence Listing for this application is labeled “CUHK.221.xml” which was created on Aug. 29, 2023 and is 5,320 bytes. The entire contents of the sequence listing is incorporated herein by reference in its entirety.

### BACKGROUND OF THE INVENTION

Liver cancer is the fourth most common cancer worldwide with approximately 900,000 cases diagnosed annually [1]. It causes over 830,000 deaths annually, making liver cancer the third most common cause of cancer death worldwide [1]. Hepatocellular carcinoma (HCC) is the most common form of liver cancer and accounts for about 90% of the cases. HCC is challenging to manage. Most HCC patients are diagnosed in the advanced stage when patients are not eligible for surgical treatments. This leads to a generally poor prognosis. a 5-year survival rate of 36% in those diagnosed in early stages and less than 13% in late stages [2]. To date, three multikinase inhibitors, sorafenib, regorafenib and lenvatinib, have been approved by the FDA for advanced HCC. However, the benefit of multikinase inhibitors has been modest, with the median survival prolonged by a few months [3, 4].

Currently available biomarkers for HCC are either non-specific or lack treatment indication. Immune checkpoint inhibitors have shown promising effects in different types of cancers; however, their response rate in HCC is low and only effective in some patients [5]. Therefore, there is a pressing need for effective prognostic markers and treatment regimens.

### BRIEF SUMMARY OF THE INVENTION

The subject invention relates to methods of using effective prognostic markers to diagnose and treat patients with liver diseases. The present invention provides the use of ADRM1/Rpn13 spliced variant ΔEx9 (ADRM1-ΔEx9) as a biomarker for prediction of prognosis in hepatocellular carcinoma (HCC) patients. By assessing the expression of the ADRM1-ΔEx9 isoform in a patient biopsy or resected tumor, prognostic outcome are predicted. In certain embodiments, ADRM1-ΔEx9 can be a molecular marker to predict the response of HCC cells to PARP1 inhibitors, such as, for example, Olaparib, Rucaparib, Niraparib, Talazoparib, or any combination thereof.

In certain embodiments, Olaparib, Rucaparib, Niraparib, Talazoparib, or other PARP1 inhibitors inhibit the DNA repair enzyme poly ADP ribose polymerase (PARP). It is commonly used for cancer patients with a documented deleterious or suspected deleterious germline mutation in BRCA1 or BRCA2 or a positive laboratory test for genomic instability called homologous recombination DNA-repair deficiency (HRD). In certain embodiments, the therapeutic use of Olaparib, Rucaparib, Niraparib, Talazoparib, or any combination thereof can be used to treat other cancer types, including, for example, breast cancer, ovarian cancer, fallopian tube cancer, peritoneal cancer, pancreatic cancer, and prostate cancer. However, the sensitivity of cancer cells to Olaparib is independent of BRCA mutations or HRD status. In certain embodiments, ADRM1-ΔEx9 is a binding partner of BRCA1 Associated Protein 1 (BAP1). In certain embodiments, increased ADRM1-ΔEx9 expression correlates with enhanced anti-tumor efficacy of Olaparib, Rucaparib, Niraparib, Talazoparib, or other PARP1 inhibitors in patient-derived HCC organoids. In certain embodiments, Olaparib, Rucaparib, Niraparib, Talazoparib, or other PARP1 inhibitors can be used to treat HCC tumors with a high expression of ADRM1-ΔEx9. In certain embodiments, ADRM1-ΔEx9 was overexpressed in 63.9% of HCC patients.

In certain embodiments, an increased expression ADRM1-ΔEx9 caused a reduction in the level of tumor suppressor protein FBXW7. In certain embodiments, deubiquitinase BAP1 is a binding partner to ADRM1-ΔEx9.

### BRIEF DESCRIPTION OF THE DRAWINGS

FIGS. 1A-1I Common ADRM1-ΔEx9 upregulation in HCC correlates with shorter patient survival. (FIG. 1A) Maps of unannotated ADRM1 isoforms and its annotated canonical counterparts shown with exon arrangement and spliced sites highlighted. Exon 9 skipping caused a truncation in the C-terminus of the ADRM1 protein. (FIG. 1B) AlphaFold 2 prediction shows that ADRM1-ΔEx9 isoform has a distinct C-terminus when compared with ADRM1-full length (FL). (FIG. 1C) Juncture flanking Taqman assays showed that ADRM1-ΔEx9 is significantly upregulated in HCC tumor compared with matched adjacent non-tumor. (FIG. 1D) ADRM1-ΔEx9, but not ADRM1-FL, is frequently upregulated in primary HCC (FIG. 1E) Pairwise analysis showed that dominance of ADRM1 variants was changed significantly in tumor and adjacent non-tumor tissue. (FIG. 1F and FIG. 1H) ADRM1-ΔEx9 is significantly associated with overall survival and disease-free survival of HCC patients, but ADRM1-FL is not. (FIG. 1G and FIG. 11) Cox regression multivariate analysis showed that ADRM1-ΔEx9 expression could serve as an independent prognostic biomarker for both overall survival and disease-free survival.

FIGS. 2A-2E ADRM1-ΔEx9, but not ADRM1-FL, conferred growth advantages to L02 cells. (FIG. 2A) Immunoblots showed that flag-ADRM1-FL and -ΔEx9 were successfully overexpressed in L02 cells. (FIGS. 2B-2D) Overexpression of ADRM1-ΔEx9, but not ADRM1-FL, promoted proliferation (FIG. 2B), colony forming (FIG. 2C) and migration (FIG. 2D) of L02 cells. (FIG. 2E) Subcutaneous tumor models showed that ADRM1-ΔEx9 enhanced the proliferation of L02 cells in vivo, whereas the effect from ADRM1-FL overexpression was not apparent.

FIGS. 3A-3E ADRM1-ΔEx9 confers growth advantages to liver organoids. Two liver organoids, namely liver organoid 1 (Liver.Org 1) and liver organoid 2 (Liver.Org 2), were generated from human liver tissue. Empty vector (EV), ADRM1-FL and -ΔEx9 were ectopically expressed in liver organoids through viral transduction. (FIG. 3A) Quantitative results of H&E staining showed that the proportion of organoids with tumor-like morphology is significantly higher in ADRM-ΔEx9 overexpressing group when compared with that of vector control and ADRM1-FL liver organoids. (FIG. 3B) Representative images of H&E staining showed that vector control and ADRM1-FL liver organoids maintained in single-layered compartments, whereas ADRM1-ΔEx9 exhibited distinct morphologies where thickened walls invaginating into the lumens were seen and pleomorphic malignant features present. (FIG. 3C) EV and ADRM1-FL overexpressing organoids stop growing within 1 month after viral transduction, whereas ADRM1-ΔEx9 overexpressing liver organoids can be proliferative for more than 3 months. (FIG. 3D) Representative brightfield images demonstrated that 1 month after viral infection EV and ADRM1-FL exhibited dispersed particles, whereas ADRM1-ΔEx9 organoids became compact and solid spheroids. (FIG. 3E) Staining of Calcein AM (CalAM, viability) and Ethidium Homodimer-2 (EthD, death) in liver organoids 1 month after viral transduction. The result demonstrated that ectopic expression of ADRM1-ΔEx9 significantly enhanced the viability of liver organoids.

FIGS. 4A-4F ADRM1-ΔEx9 is essential for HCC proliferation and survival. (FIG. 4A) Result from qPCR showed that ADRM1-ΔEx9 is specifically knocked down in HKCI-10 and Hep3B cells. (FIGS. 4B-4C) Knockdown of ADRM1-ΔEx9 profoundly suppressed the proliferation (FIG. 4B) and colony formation (FIG. 4C) of HKCI-10 and Hep3B cells. (FIG. 4D) TUNEL staining demonstrated that knockdown of ADRM1-ΔEx9 induced massive cell death in HKCI-10 and Hep3B cells. (FIG. 4E) Phase-contrast images, EthD staining and quantitative result from Caspase 3/7 staining indicated that knockdown of ADRM1-ΔEx9 induced marked apoptosis in HCC tumor organoid H775T.Org. (FIG. 4F) Orthotopic tumor models demonstrated that knockdown of ADRM1-ΔEx9 close to diminish the growth of Hep3B in vivo.

FIGS. 5A-5I ADRM1 redirects the UPS specificity to selective degrade FBXW7. (FIG. 5A) Human Ubiquitin array indicated that knockdown of ADRM1-ΔEx9 increased FBXW7 protein level in HKCI-10 cell. (FIG. 5B) Immunoblots confirmed that ADRM1-ΔEx9 negatively regulated FBXW7 protein level in HKCI-10 and L02 cells. (FIG. 5C) Inverse correlation of FBXW7 protein and ADRM1-ΔEx9 expression in HCC primary tumor specimens, patient-derived tumor organoids and cell lines. (FIG. SD) Sequence alignment of the consensus binding motif recognized by FBXW7 on ADRM1-ΔEx9, along with those found in known FBXW7 substrates, as indicated. Exon 9 skipping in ADRM1 novel isoform creates a de novo FBXW7 consensus binding sequence. (FIGS. 5E-5F) Immunoprecipitation results proved that ADRM1-ΔEx9 directly bound to FBXW7. (FIG. 5G) Treatment of proteasome inhibitor MG132 reverted the effect of ADRM1-ΔEx9 overexpression on FBXW7 protein. (FIG. 5H) Ubiquitin pull-down assay showed that ADRM1-ΔEx9 increased the ubiquitination of FBXW7 protein. (FIG. 51) Overexpression of ADRM1-ΔEx9, but not -FL and empty vector (EV) control, abolished the FBXW7-induced cell death in L02 cells.

FIGS. 6A-6F Olaparib is effective in suppressing ADRM1-ΔEx9 highly expressed HCC. (FIGS. 6A-6B) Immunoprecipitation results proved that ADRM1-ΔEx9 directly bound to BAP1 protein, whereas the canonical binding to Uch37 was absent. (FIG. 6C) Dose-response curves to Olaparib showed that ADRM1-ΔEx9 overexpressing L02 cells are more sensitive to Olaparib. (FIG. 6D) Growth curves and representative images of subcutaneous xenografts showed that Olaparib significantly suppressed the growth of ADRM1-ΔEx9 overexpressed L02 cells in vivo, whereas the effects on empty vector and ADRM1-FL group were not prominent. (FIG. 6E) Results from qPCR showed differential expression of ADRM1-ΔEx9 and comparable ADRM1-FL expression in three HCC tumor organoids, namely H670T.Org, H775T.Org and H720T.Org. (FIG. 6F) Does-response curves to Olaparib showed that organoids highly expressed ADRM1-ΔEx9 exhibited increased sensitivity to Olaparib.

### BRIEF DESCRIPTION OF THE SEQUENCES

SEQ ID NO: 1: ADRM1-ΔEx9 Forward Primer
SEQ ID NO: 2: ADRM1-ΔEx9 Reverse Primer
SEQ ID NO: 3: ADRM1-ΔEx9 Probe
SEQ ID NO: 4: ADRM1-ΔEx9 mRNA sequence

### DETAILED DISCLOSURE OF THE INVENTION

The subject invention pertains to methods of diagnosing liver disease, aiding in the diagnosis of liver disease, treating the liver disease, and/or predicting the effectiveness of a treatment of liver disease. More specifically, the subject invention provides methods for using the ADRM1/Rpn13 spliced variant ΔEx9 (ADRM1-ΔEx9) in methods of diagnosing liver disease, aiding in the diagnosis of liver disease, treating the liver disease, and/or predicting the effectiveness of a treatment of liver disease by assessing the expression of the ADRM1-ΔEx9 isoform in a biopsy or resected tumor. In certain embodiments, a PARP1 inhibitor is administered to a subject to treat the liver disease.

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the term “about” is used, these compositions contain the stated amount of the ingredient with a variation (error range) of 0-10% around the value (X±10%). In other contexts, the term “about” is used provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

“Subject” refers to an animal, such as a mammal, for example a human. The methods described herein can be useful in both humans and non-human animals. In some embodiments, the subject is a mammal (such as an animal model of disease), and in some embodiments, the subject is a human. The terms “subject” and “patient” can be used interchangeably. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate with a liver, such as, for example, a cephalopod.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

The terms “antagonist” and “inhibitor” may be used interchangeably, and they refer to a compound having the ability to inhibit a biological function of a target protein, whether by inhibiting the activity or expression of the target protein. Accordingly, the terms “antagonist” and “inhibitor” are defined in the context of the biological role of the target protein.

In some embodiments of the invention, the method comprises administration of multiple doses of the compositions of the subject invention. The method may comprise administration of 1, 2, 3, 4, 5, 6, 7, 8, 9, 10, 15, 20, 25, 30, 35, 40, or more therapeutically effective doses of a composition of the subject invention as described herein. In some embodiments, doses are administered over the course of 1 day, 2 days, 3 days, 4 days, 5 days, 6 days, 7 days, 10 days, 14 days, 21 days, 30 days, 2 months, 3 months, 4 months, 5 months, 6 months, 7 months, 8 months, 9 months, 10 months, 11 months, 1 year, or more than 1 year. Moreover, treatment of a subject with a therapeutically effective amount of the compositions of the invention can include a single treatment or can include a series of treatments. It will also be appreciated that the effective dosage of a composition used for treatment may increase or decrease over the course of a particular treatment. Changes in dosage may result and become apparent from the results of diagnostic assays or imaging techniques for detecting tumor sizes known in the art. In some embodiments of the invention, the method comprises administration of the composition several time per day, including but not limiting to 2 times per day, 3 times per day, and 4 times per day.

“Treatment”, “treating”, “palliating” and “ameliorating” (and grammatical variants of these terms), as used herein, are used interchangeably. These terms refer to an approach for obtaining beneficial or desired results including but not limited to therapeutic benefit. A therapeutic benefit is achieved with the eradication or amelioration of one or more of the physiological symptoms associated with the underlying cancer such that an improvement is observed in the patient, notwithstanding that the patient may still be afflicted with the cancer.

As used herein, the term “cancer” refers to the presence of cells possessing abnormal growth characteristics, such as uncontrolled proliferation, immortality, metastatic potential, rapid growth and proliferation rate, perturbed oncogenic signaling, and certain characteristic morphological features.

The term “effective amount” or “therapeutically effective amount” refers to that amount of an inhibitor described herein that is sufficient to affect the intended application, including but not limited to disease treatment. The therapeutically effective amount may vary depending on the intended application (in vitro or in vivo) or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims.

### Methods of Using ADRM1/RPN13 Spliced Variant ΔEX9 to Diagnose HCC

The subject invention provides an effective and accurate method for predicting prognostic outcome of HCC patients. The method of the subject invention comprises first obtaining a biological sample from the subject. Then, measuring the expression level of ADRM1-ΔEx9 in a biological sample taken from the subject, comparing the expression level with a reference amount of ADRM1-ΔEx9 mRNA in HCC tumor, and treating the subject. In preferred embodiments, the biological sample is tissue from the primary HCC tumor.

In certain embodiments, the mRNA sequence of ADRM1-ΔEx9 is:

(SEQ ID NO: 4)
ATGACGACCTCAGGCGCGCTCTTTCCAAGCCTGGTGCCAGGCTCTCG
GGGCGCCTCCAACAAGTACTTGGTGGAGTTTCGGGCGGGAAAGATGT
CCCTGAAGGGGACCACCGTGACTCCGGATAAGCGGAAAGGGCTGGTG
TACATTCAGCAGACGGACGACTCGCTTATTCACTTCTGCTGGAAGGA
CAGGACGTCCGGGAACGTGGAAGACGACTTGATCATCTTCCCTGACG
ACTGTGAGTTCAAGCGGGTGCCGCAGTGCCCCAGCGGGAGGGTCTAC
GTGCTGAAGTTCAAGGCAGGGTCCAAGCGGCTTTTCTTCTGGATGCA
GGAACCCAAGACAGACCAGGATGAGGAGCATTGCCGGAAAGTCAACG
AGTATCTGAACAACCCCCCGATGCCTGGGGCGCTGGGGGCCAGCGGA
AGCAGCGGCCACGAACTCTCTGCGCTAGGCGGTGAGGGTGGCCTGCA
GAGCCTGCTGGGAAACATGAGCCACAGCCAGCTCATGCAGCTCATCG
GACCAGCCGGCCTTGGAGGACTGGGTGGGCTGGGGGCCCTGACTGGA
CCTGGCCTGGCCAGCTTACTGGGGAGCAGTGGGCCTCCAGGGAGCAG
CTCCTCCTCCAGCTCCCGGAGCCAGTCGGCAGCGGTCACCCCGTCAT
CCACCACCTCTTCCACCCGTGCCACCCCAGCCCCTTCTGCTCCAGCA
GCTGCCTCAGCAACTAGCCCGAGCCCCGCGCCCAGTTCCGGGAATGG
AGCCAGCACAGCAGCCAGCCCGACCCAGCCCATCCAGCTGAGCGACC
TCCAGAGCATCCTGGCCACGATGAACGTACCAGCCGGGCCAGCAGGC
GGCCAGCAAGTGGACCTGGCCAGTGTGCTGACGCCGGAGATAATGGC
TCCCATCCTCGCCAACGCGGATGTCCAGGAGCGCCTGCTTCCCTACT
TGCCATCTGGGGAGTCGCTGCCGCAGACCGCGGATGAGATCCAGAAT
ACCCTGACCTCGCCCCAGTTCCAGCAGATGTGGAAGCGTTTGCCAAA
GCCATGCAGAACAACGCCAAGCCCGAGCAGAAAGAGGGCGACACGAA
GGACAAGAAGGACGAAGAGGAGGACATGA.

In some embodiments, the biological sample is liver tissue, preferably tissue from a tumor in the liver, such as, for example, a tissue sample from a liver biopsy or resected liver. In certain embodiments, mRNA can be isolated from the tissue sample. In certain embodiments, the mRNA can be isolated using an RNA extraction kit and associated methods, such as, for example RNeasy® (Qiagen, Hilden, Germany) or Direct-zol (Zymo Research).

In certain embodiments, measuring the expression level of ADRM1-ΔEx9 in a sample taken from the subject can be determined using conventional methods, such as, for example, northern blotting, nuclease protection assays, in situ hybridization, or a one-step or two-step quantitative reverse transcriptase polymerase chain reaction (RT-qPCR). In preferred embodiments, the isolated mRNA can be reverse transcribed, according to methods known in art and the resulting cDNA can be amplified using primers specific to ADRM1-ΔEx9 and at least one probe (e.g., fluorescent probe) specific to ADRM1-ΔEx9. In certain embodiments, the forward primer is CGCGGATGAGATCCAGAATAC (SEQ ID NO: 1), the reverse primer is TCCTCCTCTTCGTCCTTCTT (SEQ ID NO: 2), and the probe is CAGTTCCAGCAGATGTGGAAGCGTTT (SEQ ID NO: 3).

In certain embodiments, the expression levels of ADRM1-ΔEx9 in a sample can be compared to reference levels. In certain embodiments, ADRM1-ΔEx9 expression can be measured in non-tumor liver tissues to establish a reference level, particularly in a non-tumor liver tissue that is adjacent to liver tumor tissue in a subject. In certain embodiments, the expression of ADRM1-ΔEx9 can normalized with 18S rRNA. In certain embodiments, the fold change of ADRM1-ΔEx9 in tumor tissue compared to non-tumor tissue can be calculated. In certain embodiments, if a subject has a fold change greater than about 3, about 3.5, about 3.8842, about 4, about 4.4523, about 5, about 10, about 15, about 20, about 25, about 30, about 40, about 50, about 60, about 70 about 75, about 80, about 90, about 95, about 100, about 105, about 110, about 113, about 115, about 120, about 125, about 150 in the tumor tissue relative to the reference level, it indicates the onset of HCC or an increased risk of HCC. In certain embodiments, when the subject has a fold change greater than about 3.8842 in the tumor tissue relative to the reference level, the subject is treated with a PARP1 inhibitor.

In certain embodiments, the method of the subject invention comprises treating a subject with HCC if the expression level of ADRM1-ΔEx9 in the biological sample is significantly higher or significantly lower than the reference level of ADRM1-ΔEx9. In preferred embodiments, the method of the subject invention comprises treating a subject with HCC if the expression level of ADRM1-ΔEx9 in the biological sample is significantly higher than the reference level of ADRM1-ΔEx9. In certain embodiments, the increase in the expression level of ADRM1-ΔEx9 indicates that the subject has poorer prognosis if the HCC is untreated, and the administration of a PARP1 inhibitor will treat the HCC. In some embodiments, an increase in the expression level of ADRM1-ΔEx9 when compared with the average amount indicates the more robust response of the subject to Olaparib.

In certain embodiments, the methods can further include communicating the subject's risk of worsening or progressing HCC.

### Methods of Treating HCC

The subject invention provides methods of treating HCC with a PARP1 inhibitor, such as, for example, Olaparib, Rucaparib, Niraparib, Talazoparib, or any combination thereof. In certain embodiments, about 1 mg to about 1000 mg, about 10 mg to about 750 mg, about 30 mg to about 500 mg, or about 300 mg of the PARP1 inhibitor can be administered to the subject. In certain embodiments, the subject can be treated daily, twice per week, weekly, biweekly, monthly, bimonthly, quarterly, twice per year, yearly, or biyearly. In certain embodiments, at least 1, 2, 3, 4, 5, 6, 7, 8, 9, or 10 doses of the PARP1 can be administered at each treatment interval (e.g., daily, twice per week, weekly, etc.). In certain embodiments, the PARP1 inhibitor can be administered while monitoring the toxicity of the PARP1 inhibitor, and the treatment can be stopped if the dose is toxic to the subject. In certain embodiments, the size and location of a tumor and the expression level of ADRM1-ΔEx9 can be measured during the treatment.

In certain embodiments, the administration of the PARP1 inhibitor can be combined with other known treatments of HCC. Known treatments of HCC include, for example, curative resection, liver transplantation, radiofrequency ablation, trans-arterial chemoembolization, radioembolization, and systemic targeted agents, such as, for example, a multi-kinase inhibitor and immune checkpoint inhibitor. In certain embodiments, the multi-kinase inhibitor is, for example, Sorafenib, Lenvatinib, Cabozantinib, Regorafenib, or any combination thereof. In certain embodiments, the immune checkpoint inhibitor is, for example, Atezolizumab, Bevacizumab, Nivolumab, or any combination thereof.

In certain embodiments, ADRM1-ΔEx9 caused a reduction in the level of tumor suppressor protein FBXW7 by creating a de novo consensus protein binding motif to FBXW7. In certain embodiments, administration of a PARP1 inhibitor increases the level of FBXW7.

### Materials and Methods

### Total RNA Extraction

Total RNA from liver tissues were extracted using Qiagen RNeasy Plus Mini Kit (Catalog Number:74034). Tissues were minced on dry ice with scalpels precooled in −80° C. freezer. Six hundred microliters of Buffer RLT Plus was added to the minced tissues. After vertexing and centrifugation, the supernatant was transferred to gDNA eliminator spin columns to eliminate genomic DNA. Six hundred microliters of 70% ethanol was added to the flow-through and mixed by pipetting up and down. The mixture was then transferred to RNeasy spin columns and centrifuged at 8,000 g for 15 seconds at room temperature. The columns were washed once with 700 μL Buffer RW1, and then twice with 700 μL Buffer RPE. The columns were spun for 2 minutes at 15,000 g in order to thoroughly dry the resin. At last, RNA was eluted from the columns with 30 μL Nuclease-free water. The purified RNA samples were placed at −80° C. for long term storage. The concentration and purity of the extracted RNA were determined by measuring optical density (OD) at 260 nm and ration of OD 260/280, respectively, using the ND-2000 UV-VIS Spectrometer.

### Reverse Transcription

SuperScript™ III First-Strand Synthesis System kit (Invitrogen, Catalog Number: 18080051) was used to conduct reverse transcription. Five hundred nanograms of RNA sample was mixed with 1 μL dNTP mix (10 mM) and 1 μL Random hexamers (50 ng/μL). The mixture was incubated at 65° C. for 5 min, and then placed on ice for at least 1 min. After mixed with 2 μL 10×RT buffer, 4 μL 25 mM MgCl₂, 2 μL 0.1 M DTT, 1 μL RNaseOUT (40 U/μL) and 1 μL SuperScript® III RT (200 U/μL), the mixture was incubated for 10 minutes at 25° C., followed by 50 minutes at 50° C. The reaction was terminated at 85° C. for 5 minutes and then chilled on ice. At last, 1 μL RNase H was added into each tube and incubated for 20 minutes to eliminate residual RNA. After diluted 10 times with Nuclease-free water, cDNA samples were placed at −20° C. for long term storage.

TaqMan-Based Quantitative Real-Time PCR (qPCR)

TaqMan™ Universal PCR Master Mix (Applied Biosystems, Catalog Number: 4304437) was used. Forward and reverse primers of ADRM1-ΔEx9 were dissolved in nuclease-free waster at concentration of 10 μM. TaqMan™ probe of ADRM1-ΔEx9 was dissolved in nuclease-free waster at a concentration of 25 μM. Expression for ADRM1-ΔEx9 and endogenous control 18S were determined in separate wells. To determine expression of ADRM1-ΔEx9, each reaction consisted of 2 μL diluted cDNA, 5 μL TaqMan™ Universal PCR Master Mix, 1 μL forward primer, 1 μL reverse primer, 0.25 μL TaqMan™ probe and 0.75 μL Nuclease-free waster. To determine expression of endogenous control 18S, each reaction was consisted of 2 μL diluted cDNA, 5 μL TaqMan™ Universal PCR Master Mix, 0.5 μL 20× TaqMan Gene Expression assay and 2.5 μL Nuclease-free waster. All assays were conducted in triplicate wells of 384-well PCR plates (Thermo Scientific, Catalog Number: AB1384). QuantStudio™ 7 Flex Real-Time PCR System (Applied Biosystems) was used to perform qPCR.

TABLE 1
Primers and Taqman assays for isoforms

| Gene | | |
|---|---|---|
| Isoforms | Primer | Sequence |
| ADRM1- | Forward | CGCGGATGAGATCCAGAATAC |
| ΔEx9 | | (SEQ ID NO: 1) |
| | Reverse | TCCTCCTCTTCGTCCTTCTT |
| | | (SEQ ID NO: 2) |
| | Probe | CAGTTCCAGCAGATGTGGAAGCGTTT |
| | | (SEQ ID NO: 3) |
| 18s rRNA | Predesigned | Thermo Fisher: |
| | assay | Hs99999901_s1 |

### EXAMPLES

Following are examples that illustrate procedures for practicing the invention. These examples should not be construed as limiting. All percentages are by weight and all solvent mixture proportions are by volume unless otherwise noted.

### Example 1—Prognostic Value of Adrm1-Δex9 in Hcc

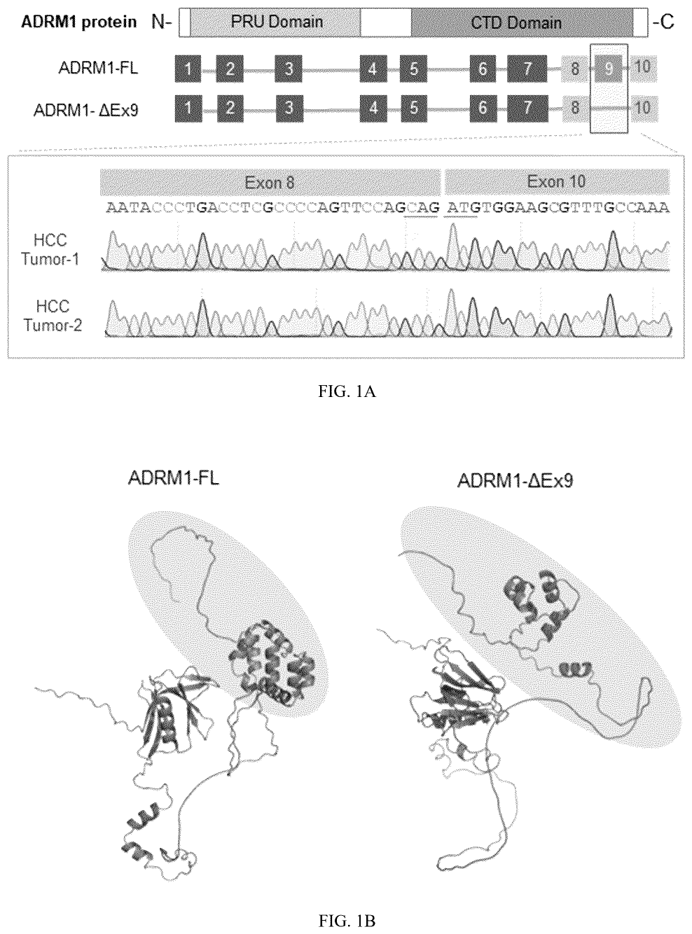

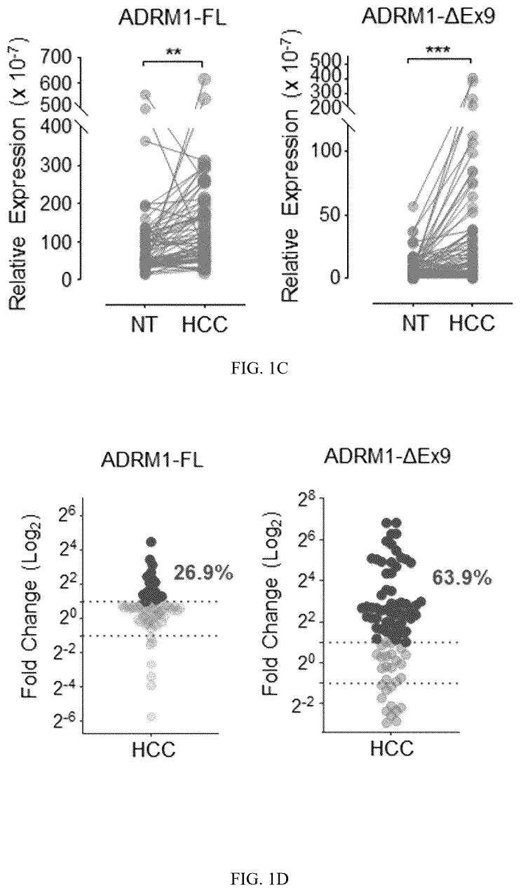

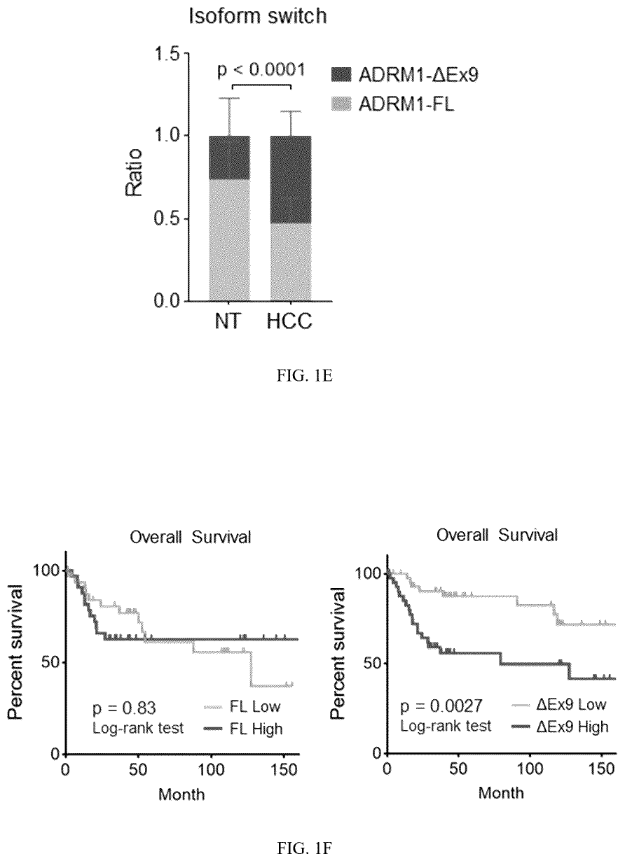

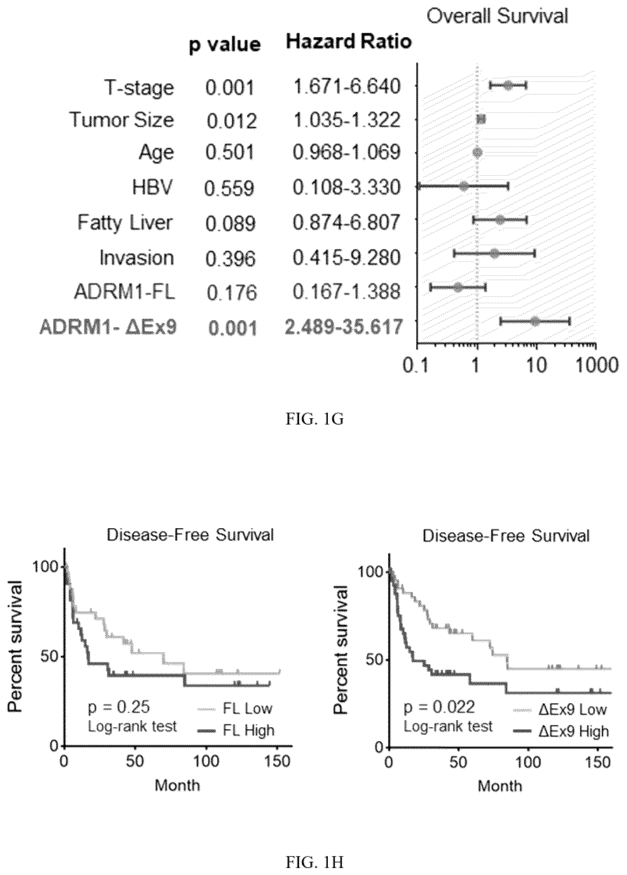

To determine the level of ADRM1-FL and ADRM1-ΔEx9 in HCC, we used juncture flanking TaqMan probes to precisely interrogate their expression in an independent cohort containing 86 pairs of HCC tumors and paired adjacent non-tumor tissue. Although both ADRM1-FL and ADRM1-ΔEx9 were significantly overexpressed in HCC tumors, pairwise comparison demonstrated that ADRM1-ΔEx9, but not ADRM1-FL, was uniformly upregulated in HCC tumors (FIG. 1C). Consistently, increase in ADRM1-ΔEx9 expression by at least 2-fold could be found in 63.9% of HCC tumors when compared to paired adjacent non-tumoral liver, whereas only 26.9% of HCC cases were associated upregulated ADRM1-FL (FIGS. 1C and 1D). We also observed a significant change in the variant dominance in HCC, where the ratio of ADRM1-ΔEx9: -FL in non-tumoral liver transformed to ADRM1-ΔEx9 dominance in tumors, suggesting a plausibly isoform-switch in driving liver oncogenesis (FIG. 1E). Notably, ADRM1-ΔEx9 correlated significantly with overall survival and disease-free survival of HCC patients (FIG. 1F), whereas ADRM1-FL did not (FIG. 1H). Cox regression multivariate analysis further confirmed that ADRM1-ΔEx9 expression could be an independent prognostic biomarker for overall and disease-free survival (FIGS. 1G and 1I). Collectively, overexpression of ADRM1-ΔEx9, but not the canonical isoform, is prevalent in HCC with prognostic importance.

### Example 2—Adrm1-Δex9 Conferred Oncogenicity to Hepatocyte Cells

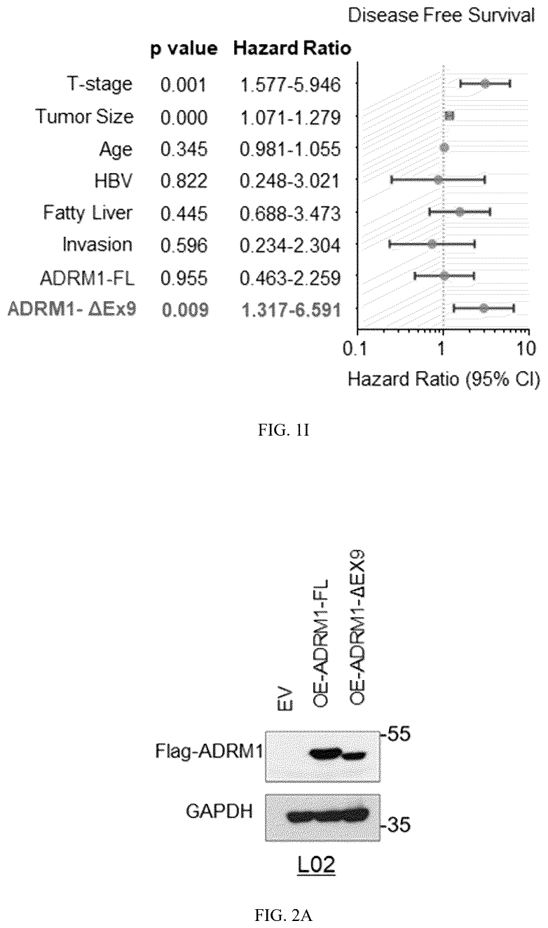

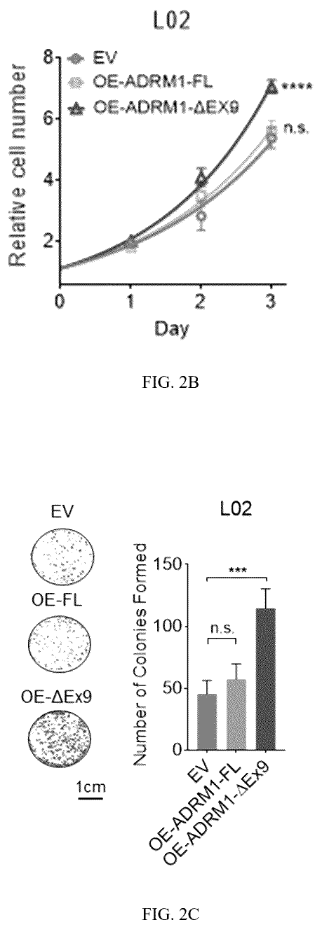

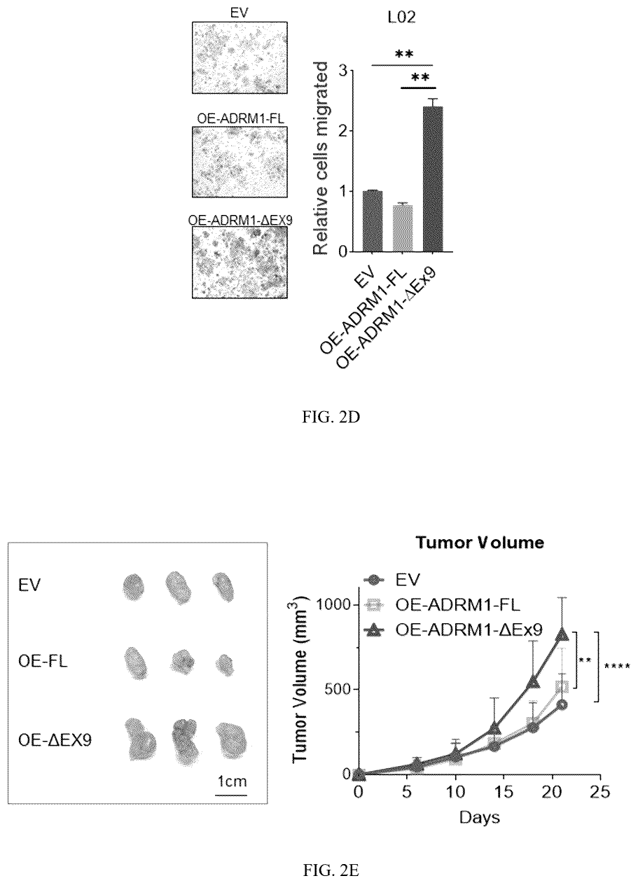

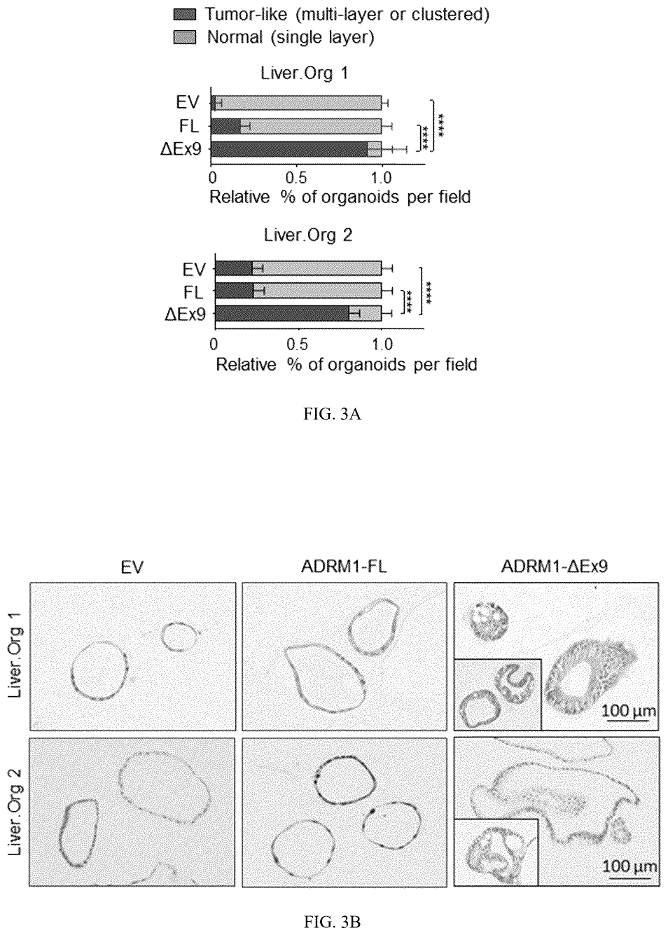

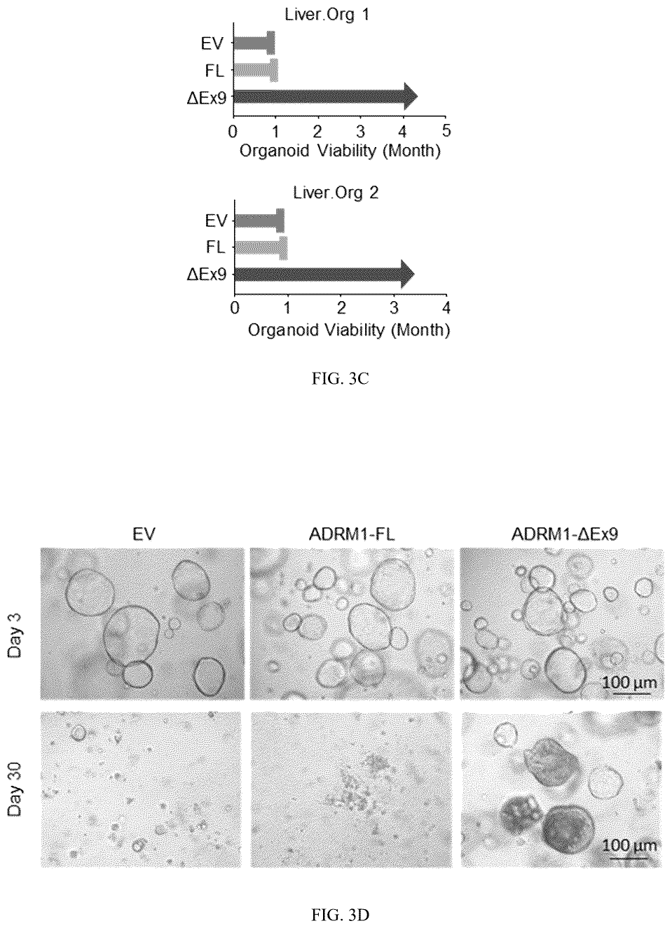

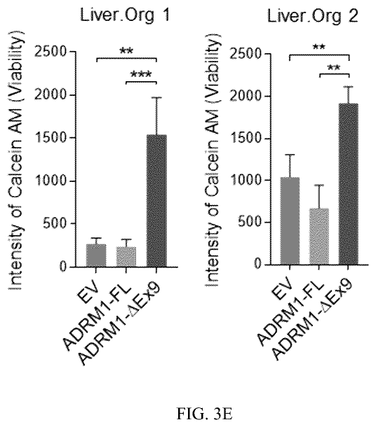

Tumor-promoting properties of ADRM1 have been presented in multiple cancer types [10, 11]. However, the roles of each splicing variant, especially the functional characteristics of the unannotated ADRM1 isoform, remain elusive in HCC. We overexpressed ADRM-FL and -ΔEx9 in human hepatocytes cell line L02 (FIG. 2A). Ectopic expression of ADRM1-ΔEx9 significantly increased cell proliferation (FIG. 2B), colony forming (FIG. 2C) and migration (FIG. 2D) in L02 cells. Consistently, subcutaneous xenografts derived from L02-ADRM1-ΔEx9 were highly proliferative compared to vector control and ADRM1-FL group (FIG. 2E). To understand the impact of ADRM1 variants on early HCC development, we developed two liver organoids, namely liver organoid 1 (Liver.Org 1) and liver organoid 2 (Liver.Org 2) from fresh non-tumoral liver tissues. ADRM1-FL or -ΔEx9 was ectopically expressed in human liver-derived non-tumoral liver organoids through viral transduction. During subculture, vector control and ADRM1-FL liver organoids maintained in single-layered compartments as reflected by the histology staining (FIGS. 3A and 3B). In contrast, distinct morphologies appeared in two ADRM1-ΔEx9-overexpressing liver organoids where thickened walls invaginating into the lumens were observed and pre-malignant features, including dysplasia, hyperchromasia, atypical and frequent mitosis, loss of polarity, and increased nuclear to cytoplasm ratio, presented (FIG. 3B). Intriguingly, non-tumoral liver organoids expressing empty vector or ADRM1-FL normally stop growing within 1 month after viral transduction (FIG. 3C), whereas ADRM1-ΔEx9 overexpressing liver organoids can be proliferative for more than 3 months (FIG. 3C). Both bright-field images and live/dead staining consistently showed that vector control and ADRM1-FL liver organoids exhibited spontaneous cell death one month after viral infection (FIGS. 3D and 3E), whereas ADRM1-ΔEx9 overexpressing liver organoids remain viable and intact (FIGS. 3D and 3E). In sum, ADRM1-ΔEx9 conferred oncogenicity to hepatocyte cells.

### Example 3—Knockdown of Adrm1-ΔEx9 Suppressed the Proliferation of Hcc Cells and Resulted in Spontaneous Cell Death

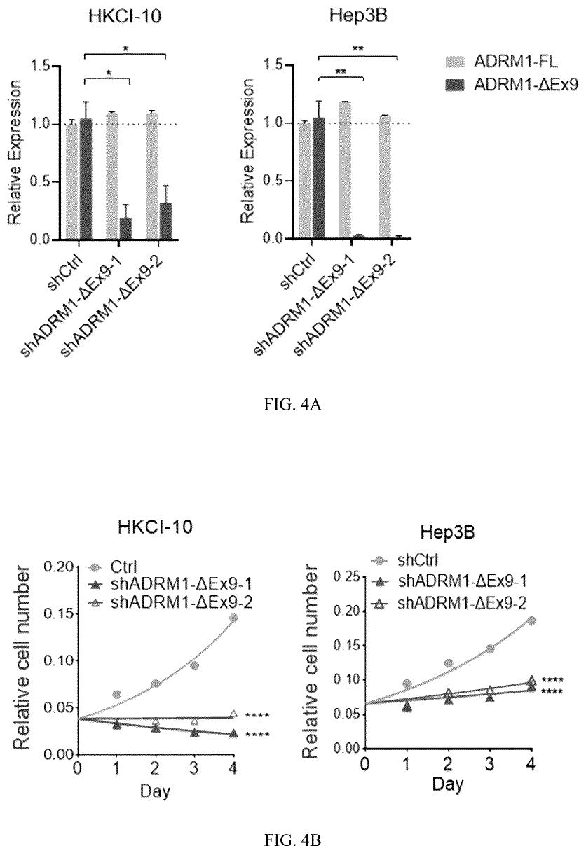

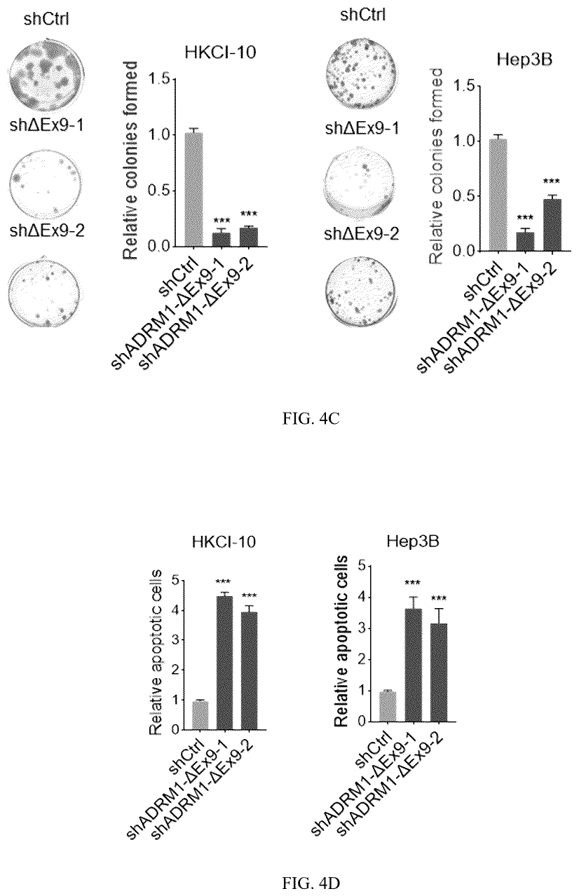

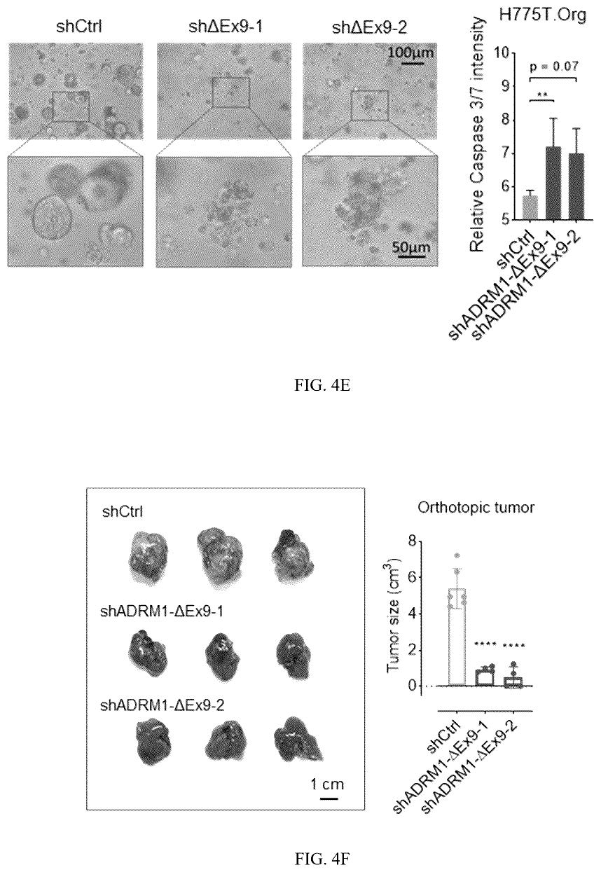

To further elucidate the biological functions of ADRM1-ΔEx9, we designed shRNAs to target the unique juncture region of exon 8 to 10 of the variant. Results from qPCR demonstrated that two shRNA sequences specially knocked down ADRM1-ΔEx9 without affecting the expression of ADRM1-FL in two HCC cell lines, Hep3B and in-house established HKCI-10 (FIG. 4A). Knockdown of ADRM1-ΔEx9 close to abolish the proliferation and colony forming capability of HKCI-10 and Hep3B cells (FIGS. 4B and 4C). This corresponded to a drastic increase in apoptosis upon knockdown of ADRM1-ΔEx9 by TUNEL staining (FIG. 4D). The phenotype was further substantiated in patient-derived HCC tumor organoids, H775T.Org. Compared to shCtrl tumor organoids which displayed uniform and compact spheroids (FIG. 4E), H775T.Org exhibited marked change with dark disintegrating spheroids and dispersed particles upon ΔEx9 knockdown (FIG. 4E). Consistently, staining of Ethidium homodimer-2 (EthD) and Caspase 3/7 indicated that knockdown of ADRM1-ΔEx9 induced pronounced cell death in tumor organoids (FIG. 4E). To understand the biology of ADRM1-ΔEx9 in a more physiological relevant tumor-microenvironment, we orthotopically injected Hep3B-shCtrl or shADRM1 cells into mice liver. In line with the observations in cell lines and organoids, knockdown of ADRM1-ΔEx9 nearly abrogated the growth of orthotopic tumor (FIG. 4F). These results demonstrated that ADRM1-ΔEx9 is essential for proliferation and survival of HCC cells.

### Example 4—Adrm1-ΔEx9 Redirected the Ups Specificity in Hcc

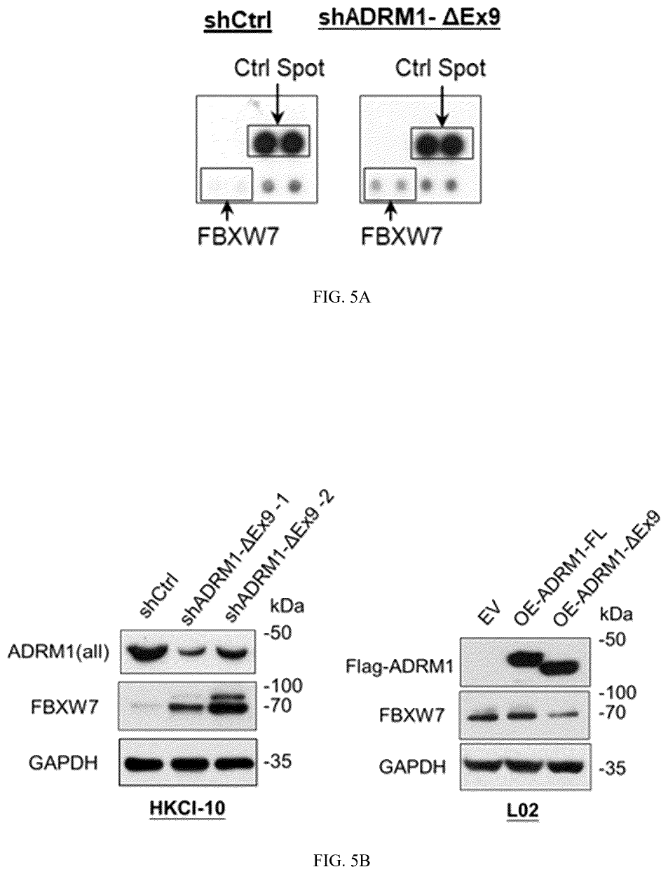

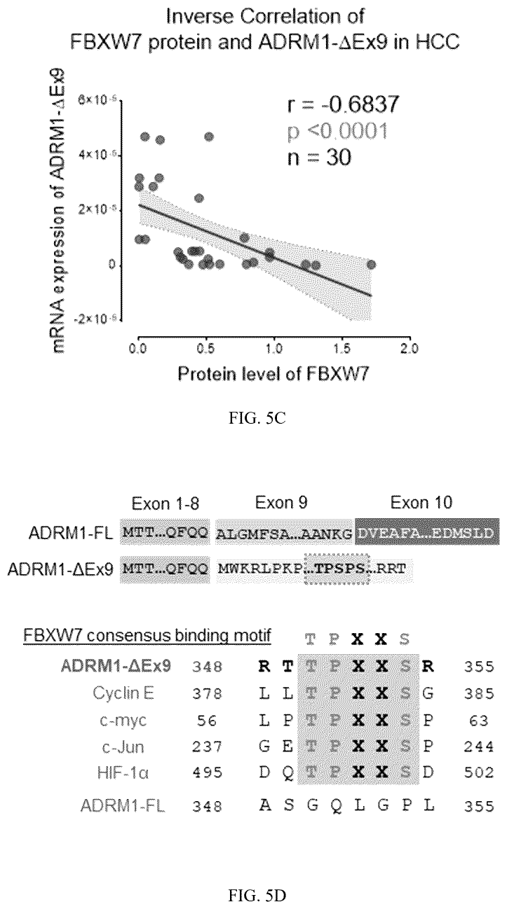

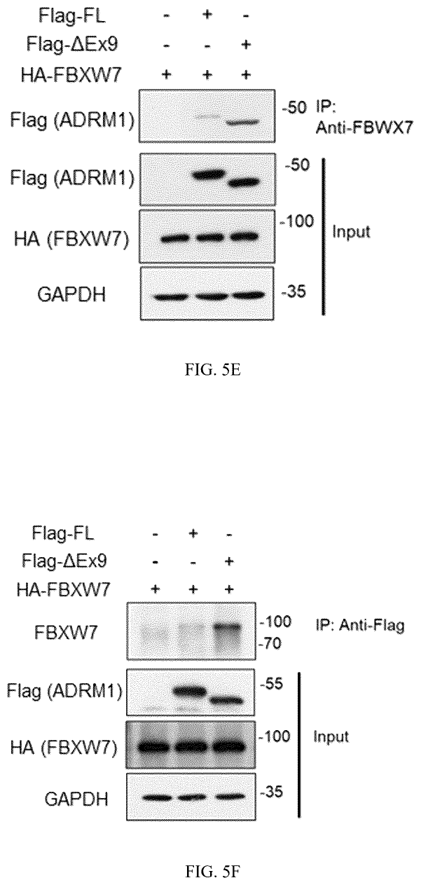

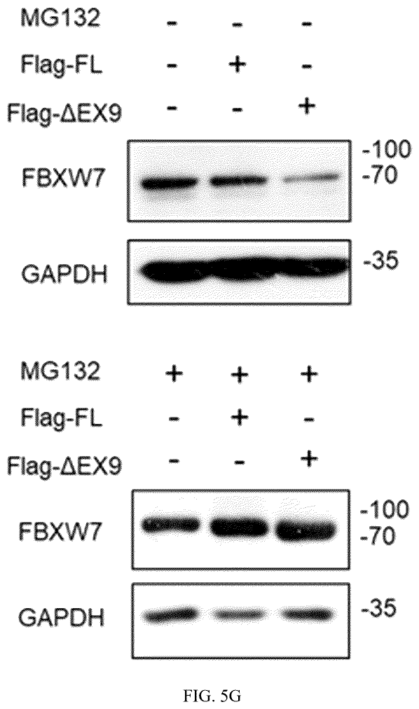

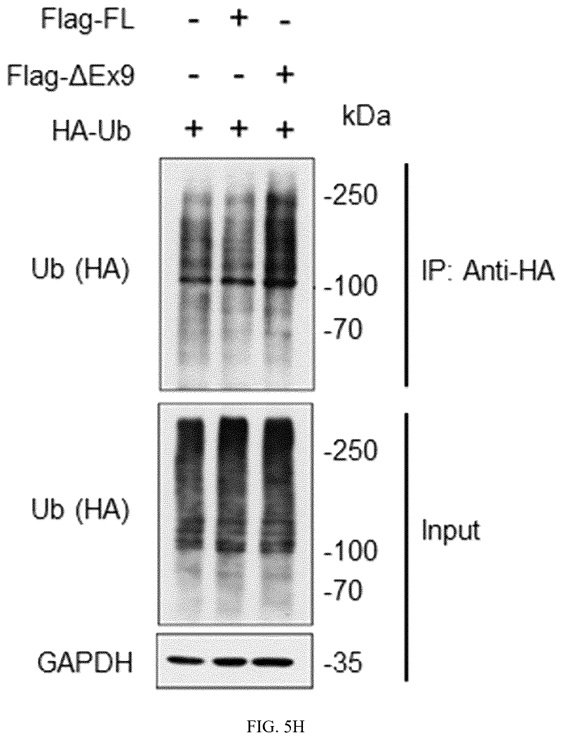

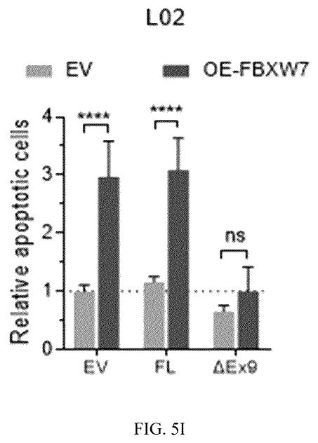

To further delineate the mechanism underlying ADRM1-ΔEx9 biology, we turned to the functional domains of ADRM1 protein. ADRM1 has two functional motifs. The N-terminal PRU domain is responsible for recognizing polyubiquitin chains [7], while the C-terminal domain directly binds to and activates deubiquitinase Uch37 [8, 13]. Alphafold2 prediction suggested that exon 9 skipping in ADRM1-ΔEx9 results in a conformational change to the C-terminus domain (FIG. 1B), that may affect the UPS. We therefore performed Proteome Profiler Human Ubiquitin Array to detect the protein level of a series of functionally important ubiquitin targets upon ADRM1-ΔEx9 knockdown. The result revealed that knockdown of ADRM1-ΔEx9 increased the level of a classic tumor suppressor protein, FBXW7 (FIG. 5A). FBXW7 is a member of the F-box protein family, which functions as a substrate recognition subunit of the SCF (SKP1/CUL1/F-box protein) E3 ubiquitin ligase [14]. It promotes the ubiquitination and degradation of several oncoproteins, including Cyclin E [15], Myc [16], c-Jun [17], and HIF-la [18], to suppress tumor cell growth and survival. Our immunoblotting concurred with Ubiquitin Array finding in demonstrating that knockdown of ADRM1-ΔEx9, but not -FL variant, increased the amount of endogenous FBXW7 protein (FIG. 5B) in HKCI-10 cells. In contrast, ectopic expression of ADRM1-ΔEx9 in hepatocyte cells L02 downregulated the protein expression of FBXW7 (FIG. 5B). To affirm the regulatory role of ADRM1-Ex9, we assessed the association between ADRM1-ΔEx9 expression and FBXW7 protein in 17 primary HCC tumor specimens and 13 patient-derived HCC tumor organoids or cell lines. The result demonstrated a significant reverse correlation between FBXW7 protein and ADRM1-ΔEx9 level with a Pearson coefficient value of −0.6837 (FIG. 5C). These results implicated that ADRM1-ΔEx9 is a modulator of FBXW7 protein in HCC.

### Example 5—Exon 9 Skipping Creates a De Novo Fbxw7 Binding Site

We next conducted an experiment to understand the role of ADRM1-ΔEx9 in FBXW7 regulation. FBXW7 is an E3 ligase that binds to a broad range of oncogenic substrates through a consensus binding motif -TPXXS- for polyubiquitination and subsequent degradation [19]. Recent studies show that a few binding partners of FBXW7, instead of being degraded, disrupt FBXW7 dimerization and promote FBXW7 self-ubiquitination and degradation [19, 20]. Intriguingly, in silico analysis revealed that exon 9 skipping in ADRM1-ΔEx9 creates a de novo FBXW7 consensus binding motif (-TPSPS-) within the codons 350-354 of the c-terminus (FIG. 5D), suggesting the potential direct interaction between these two proteins. To verify this hypothesis, we co-transfected flag-ADRM1-ΔEx9 and HA-FBXW7 in 293T cells with flag-ADRM1-FL included as a negative control. Results from immunoprecipitation showed that ADRM1-ΔEx9, but not ADRM1-FL, directly bound to FBXW7 (FIGS. 5E and 5F). Previous studies have shown that FBXW7 is subject to self-ubiquitinase while in the monomeric form, followed by degradation [21]. Indeed, we observed that treatment of proteasome inhibitor MG132 reverted the reduction of FBXW7 protein in ADRM1-ΔEx9 overexpressing cells (FIG. 5G). Ectopic expression of ADRM1-ΔEx9 increased the polyubiquitination of FBXW7, whereas total ubiquitination level in the whole cell lysate was not affected (FIG. 5H). Moreover, presence of ADRM-ΔEx9, but not the FL isoform, reduced FBXW7-induced cell death (FIG. 5I). Collectively, ADRM1-ΔEx9 counteracts the function of tumor suppressor protein FBXW7 in HCC through direct interaction and subsequent enhancement of FBXW7 polyubiquitination.

### Example 6—Parp Inhibitor Olaparib Antagonizes Adrm1-ΔEx9-Driven Hcc Growth

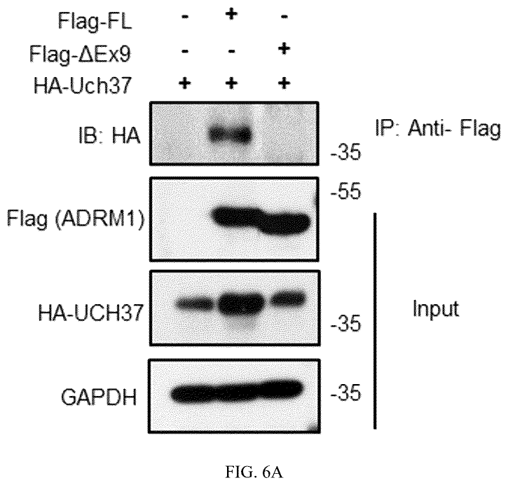

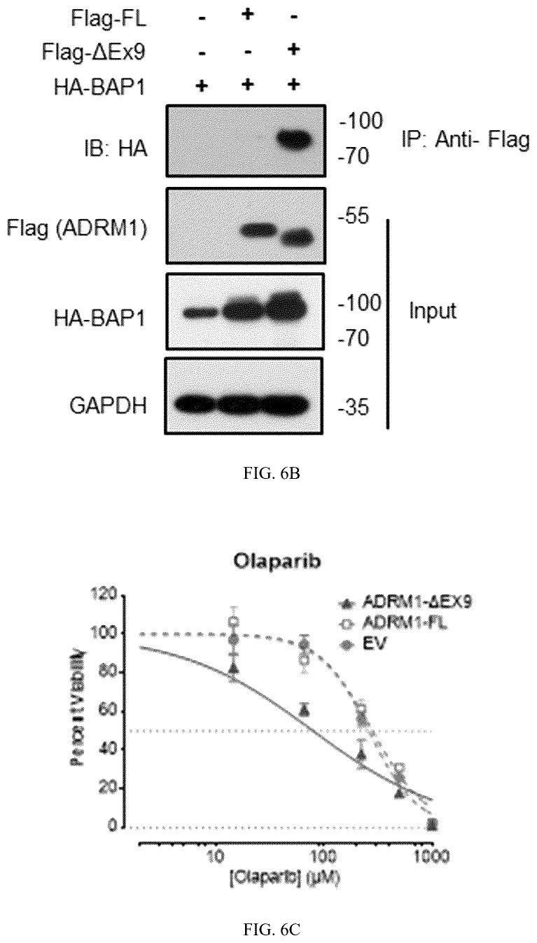

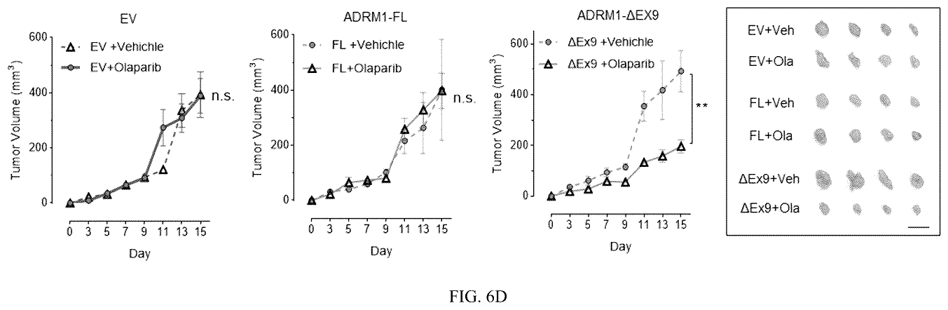

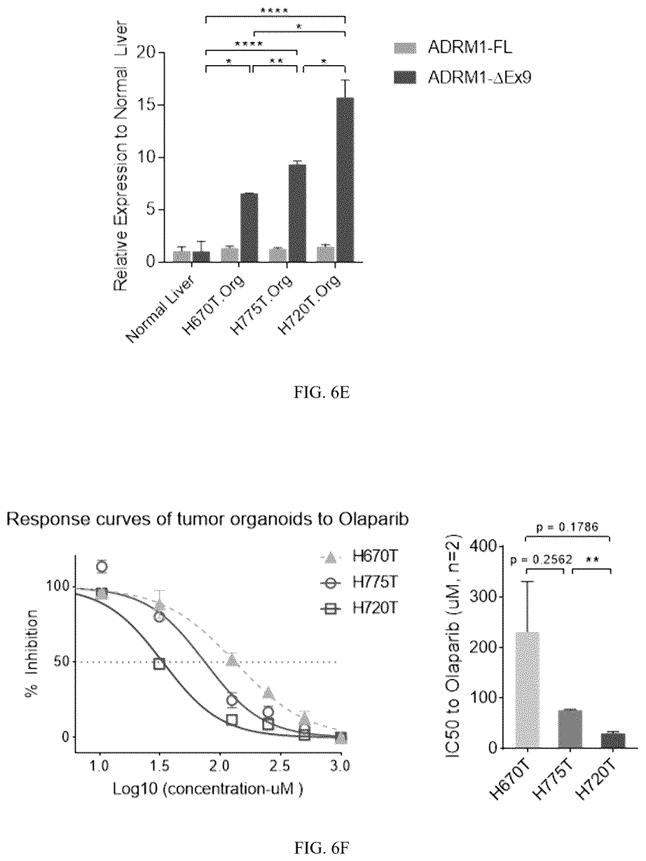

Having defined the substrate of ADRM1-ΔEx9, we further studied the mechanism by which ADRM1-ΔEx9 redirects proteasome specificity. It is known that ADRM1 mediates specific proteasomal degradation mainly by two mechanisms: (1) activating deubiquitinating enzyme Uch37 to modulate the outcome of ubiquitination [8, 13] and (2) recognizing proteins with K48-linked polyubiquitin chains through the N-terminal PRU domain [7]. As ADRM1-ΔEx9 preserves the intact PRU domain, we postulated that the rewired UPS specificity was resulted from the altered C-terminus. Indeed, immunoprecipitation assay showed that classical binding of ADRM1-ΔEx9 to deubiquitinase Uch37 is not detected (FIG. 6A), although such an association is maintained for the ADRM1-FL (FIG. 6A). It suggested the likely change in deubiquitinase partner with ADRM1-ΔEx9 that underscored the biological phenotypes and the degradation of FBXW7. We therefore assessed the interaction of ADRM1-ΔEx9 with a panel of UPS associated or cancer relevant deubiquitinases, including Uch1, Uch3, BAP1, Rpn11, USP14, A20 and USP28. Intriguingly, result from immunoprecipitation revealed that BRCA1-Associated Protein 1 (BAP1) is the unique binding partner for ADRM1-ΔEx9 (FIG. 6B). BAP1 is a ubiquitin carboxy-terminal hydrolase that coordinates BRCA1, BARD1 and RAD51 to mediate the homologous recombination DNA repair. Previous studies have shown that BAP1-altered tumors exhibited a superior response to DNA damaging agents, such as poly-ADP ribose (PARP) inhibitors [22]. We evaluated the response of ADRM1-overexpressing cells to Olaparib, one of the most widely used PARP inhibitors in clinic. Compared with the effect seen on vector control and ADRM1-FL expressing cells which exhibited IC50 values of 277.6±31.2 μM and 247.2 f 33.0 μM, respectively, ADRM1-ΔEx9 overexpressing L02 cells were more sensitive to Olaparib with an IC50 value of 123.1±55.9 μM (FIG. 6C). Moreover, Olaparib at 100 mg/kg/day significantly suppressed the growth of ΔEx9-derived subcutaneous xenografts (FIG. 6D), whereas its effects on vector control and ADRM1-FL groups were not apparent (FIG. 6D). To further test if endogenous level of ADRM1-ΔEx9 would affect the response of HCC cells to Olaparib, we selected three patient-derived HCC tumor organoids, namely H670T.Org, H775T.Org and H720T.Org. These tumor organoids expressed differential levels of ADRM1-ΔEx9 and comparable level of ADRM1-FL (FIG. 6E). CellTiter-Glo assays revealed that H720T. Org with the most abundant ADRM1-ΔEx9 expression had the highest sensitivity to Olaparib, whereas H670T.Org which lowly expressed ADRM1-ΔEx9 presented the least response (FIG. 6F). Taken together, the result indicated that Olaparib can be used to target HCC tumors that highly expressed ADRM1-ΔEx9.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXEMPLARY EMBODIMENTS

Embodiment 1. A method for treating hepatocellular carcinoma (HCC) in a subject, the method comprising:
(a) obtaining a biological sample from the subject;
(b) measuring the expression level of ADRM1-ΔEx9 in the biological sample;
(c) comparing the expression level obtained in step (b) with a reference level of ADRM1-ΔEx9 mRNA; and
(d) treating the HCC in the subject if the expression level of ADRM1-ΔEx9 in the biological sample is significantly higher or significantly lower than the reference level of ADRM1-ΔEx9.
   Embodiment 2. The method of embodiment 1, wherein the biological sample is tissue of a primary HCC tumor of the subject.
   Embodiment 3. The method of embodiment 1, wherein measuring the expression level of ADRM1-ΔEx9 comprises northern blotting, a nuclease protection assay, in situ hybridization, or quantitative reverse transcriptase polymerase chain reaction (RT-qPCR).
   Embodiment 4. The method of embodiment 1, wherein step (d) comprises treating the HCC in the subject if the expression level of ADRM1-ΔEx9 in the biological sample is significantly higher than the reference level of ADRM1-ΔEx9.
   Embodiment 5. The method of embodiment 1, wherein the expression level of ADRM1-ΔEx9 in the biological sample is at least 3.8842 fold greater than the reference level of ADRM1-ΔEx9.
   Embodiment 6. The method of embodiment 1, wherein the treating the HCC in the subject comprises administering a PARP1 inhibitor to the subject.
   Embodiment 7. The method of embodiment 6, wherein about 300 mg of the PARP1 inhibitor is administered to the subject.
   Embodiment 8. The method of embodiment 6, wherein the PARP1 inhibitor is administered at least two times per day.
   Embodiment 9. The method of embodiment 6, wherein the PARP1 inhibitor is Olaparib, Rucaparib, Niraparib, Talazoparib, or any combination thereof.
   Embodiment 10. The method of embodiment 8, wherein the PARP1 inhibitor is administered for about 2 days to about two years.

### REFERENCES

1. Rumgay H, Arnold M, Ferlay J, Lesi O, Cabasag C J, Vignat J, Laversanne M, McGlynn K A, Soerjomataram I: Global burden of primary liver cancer in 2020 and predictions to 2040. J Hepatol 2022, 77(6):1598-1606.
2. Llovet J M, Kelley R K, Villanueva A, Singal A G, Pikarsky E, Roayaie S, Lencioni R, Koike K, Zucman-Rossi J, Finn R S: Hepatocellular carcinoma. Nat Rev Dis Primers 2021, 7(1):6.
3. Kudo M, Finn R S, Qin S, Han K H, Ikeda K, Piscaglia F, Baron A, Park J W, Han G, Jassem J et al: Lenvatinib versus sorafenib in first-line treatment of patients with unresectable hepatocellular carcinoma: a randomised phase 3 non-inferiority trial. Lancet 2018, 391(10126):1163-1173.
4. Llovet J M, Ricci S, Mazzaferro V, Hilgard P, Gane E, Blanc J F, de Oliveira ΔC, Santoro A, Raoul J L, Forner A et al: Sorafenib in advanced hepatocellular carcinoma. N Engl J Med 2008, 359(4):378-390.
5. Sangro B, Sarobe P, Hervas-Stubbs S, Melero I: Advances in immunotherapy for hepatocellular carcinoma. Nat Rev Gastroenterol Hepatol 2021, 18(8):525-543.
6. Rock K L, Gramm C, Rothstein L, Clark K, Stein R, Dick L, Hwang D, Goldberg ΔL: Inhibitors of the proteasome block the degradation of most cell proteins and the generation of peptides presented on MHC class I molecules. Cell 1994, 78(5):761-771.
7. Husnjak K, Elsasser S, Zhang N, Chen X, Randles L, Shi Y, Hofmann K, Walters K J, Finley D, Dikic I: Proteasome subunit Rpn13 is a novel ubiquitin receptor. Nature 2008, 453(7194):481-488.
8. Vander Linden R T, Hemmis C W, Schmitt B, Ndoja A, Whitby F G, Robinson H, Cohen R E, Yao T, Hill C P: Structural basis for the activation and inhibition of the UCH37 deubiquitylase. Mol Cell 2015, 57(5):901-911.
9. Chen H, Gao F, He M, Ding X F, Wong ΔM, Sze S C, Yu ΔC, Sun T, Chan A W, Wang X et al: Long-Read RNA Sequencing Identifies Alternative Splice Variants in Hepatocellular Carcinoma and Tumor-Specific Isoforms. Hepatology 2019, 70(3):1011-1025.
10. Wang J, Wu X, Dai W, Li J, Xiang L, Tang W, Lin J, Zhang W, Liu G, Yang Q et al: The CCDC43-ADRM1 axis regulated by YY1, promotes proliferation and metastasis of gastric cancer. Cancer Lett 2020, 482:90-101.
11. Ning Z, Wu Z, Zhang F, Yang M, Lu Z, Yu B, Long F, Guo Y, Yang K, Hu G et al: GMEB2 Promotes the Growth of Colorectal Cancer by Activating ADRM1 Transcription and NF-kappaB Signalling and Is Positively Regulated by the m(6)A Reader YTHDF1. Cancers (Basel) 2022, 14(24).
12. Wu W, Zhong J, Chen J, Niu P, Ding Y, Han S, Xu J, Dai L: Prognostic and Therapeutic Significance of Adhesion-regulating Molecule 1 in Estrogen Receptor-positive Breast Cancer. Clin Breast Cancer 2020, 20(2):131-144 e133.
13. Lu X, Nowicka U, Sridharan V, Liu F, Randles L, Hymel D, Dyba M, Tarasov S G, Tarasova N I, Zhao X Z et al: Structure of the Rpn13-Rpn2 complex provides insights for Rpn13 and Uch37 as anticancer targets. Nat Commun 2017, 8:15540.
14. Yeh C H, Bellon M, Nicot C: FBXW7: a critical tumor suppressor of human cancers. Mol Cancer 2018, 17(1):115.
15. Minella ΔC, Welcker M, Clurman B E: Ras activity regulates cyclin E degradation by the Fbw7 pathway. Proc Natl Acad Sci USA 2005, 102(27):9649-9654.
16. King B, Trimarchi T, Reavie L, Xu L, Mullenders J, Ntziachristos P, Aranda-Orgilles B, Perez-Garcia A, Shi J, Vakoc C et al: The ubiquitin ligase FBXW7 modulates leukemia-initiating cell activity by regulating MYC stability. Cell 2013, 153(7):1552-1566.
17. Wei W, Jin J, Schlisio S, Harper J W, Kaelin W G, Jr.: The v-Jun point mutation allows c-Jun to escape GSK3-dependent recognition and destruction by the Fbw7 ubiquitin ligase. Cancer Cell 2005, 8(1):25-33.
18. Cassavaugh J M, Hale S A, Wellman T L, Howe A K, Wong C, Lounsbury K M: Negative regulation of HIF-1alpha by an FBW7-mediated degradation pathway during hypoxia. J Cell Biochem 2011, 112(12):3882-3890.
19. Welcker M, Clurman B E: FBW7 ubiquitin ligase: a tumour suppressor at the crossroads of cell division, growth and differentiation. Nat Rev Cancer 2008, 8(2):83-93.
20. Lan H, Tan M, Zhang Q, Yang F, Wang S, Li H, Xiong X, Sun Y: LSD1 destabilizes FBXW7 and abrogates FBXW7 functions independent of its demethylase activity. Proc Natl Acad Sci USA 2019, 116(25):12311-12320.
21. Welcker M, Larimore E A, Swanger J, Bengoechea-Alonso M T, Grim J E, Ericsson J, Zheng N, Clurman B E: Fbw7 dimerization determines the specificity and robustness of substrate degradation. Genes Dev 2013, 27(23):2531-2536.
22. Pena-Llopis S, Vega-Rubin-de-Celis S, Liao A, Leng N, Pavia-Jimenez A, Wang S, Yamasaki T, Zhrebker L, Sivanand S, Spence P et al: BAP1 loss defines a new class of renal cell carcinoma. Nat Genet 2012, 44(7):751-759.

We claim:

1. A method for treating hepatocellular carcinoma (HCC) in a subject, the method comprising:
(a) obtaining a biological sample from the subject;
(b) measuring the expression level of ADRM1-ΔEx9 in the biological sample;
(c) comparing the expression level obtained in step (b) with a reference level of ADRM1-Δex9 mRNA; and
(d) treating the HCC in the subject if the expression level of ADRM1-ΔEx9 in the biological sample is significantly higher or significantly lower than the reference level of ADRM1-ΔEx9.

2. The method of claim 1, wherein the biological sample is tissue of a primary HCC tumor of the subject.

3. The method of claim 1, wherein measuring the expression level of ADRM1-ΔEx9 comprises northern blotting, a nuclease protection assay, in situ hybridization, or quantitative reverse transcriptase polymerase chain reaction (RT-qPCR).

4. The method of claim 1, wherein step (d) comprises treating the HCC in the subject if the expression level of ADRM1-ΔEx9 in the biological sample is significantly higher than the reference level of ADRM1-ΔEx9.

5. The method of claim 1, wherein the expression level of ADRM1-ΔEx9 in the biological sample is at least 3.8842 fold greater than the reference level of ADRM1-ΔEx9.

6. The method of claim 1, wherein the treating the HCC in the subject comprises administering a PARP1 inhibitor to the subject.

7. The method of claim 6, wherein about 300 mg of the PARP1 inhibitor is administered to the subject.

8. The method of claim 6, wherein the PARP1 inhibitor is administered at least two times per day.

9. The method of claim 6, wherein the PARP1 inhibitor is Olaparib, Rucaparib, Niraparib, Talazoparib, or any combination thereof.

10. The method of claim 8, wherein the PARP1 inhibitor is administered for about 2 days to about two years.
